(19) United States
(12) Patent Application Publication
(10) Pub. No.: US 2017/0043344 A1
(43) Pub. Date: Feb. 16, 2017

(54) POLYMERASE CHAIN REACTION DEVICE AND POLYMERASE CHAIN REACTION METHOD

(71) Applicant: Seiko Epson Corporation, Tokyo (JP)

(72) Inventor: Taro AOKI, Yazawa (JP)

(21) Appl. No.: 15/232,064

(22) Filed: Aug. 9, 2016

(30) Foreign Application Priority Data
Aug. 11, 2015 (JP) 2015-158761

(51) Int. Cl. B01L 7/00; C12Q 1/68; C12P 19/34

(52) U.S. Cl. CPC B01L 7/525; C12P 19/34; C12Q 1/686; B01L 2400/0415; B01L 2300/18; B01L 2300/0609; B01L 2200/14

(57) ABSTRACT

A PCR device, in which a vessel is filled with a liquid which has a lower specific gravity than a reaction mixture and is immiscible with the reaction mixture, and a control section drives and controls a first heating section and a second heating section so that the liquid in an upper part in the vessel is brought to a first temperature and the liquid in a lower part is brought to a second temperature which is lower than the first temperature, and also drives and controls an electric field generation section to generate an electric field between a lower electrode and an upper electrode so that the reaction mixture in a spherical shape in the liquid moves up and down repeatedly between the upper part and the lower part of the liquid by a Coulomb force due to the electric field.

### CROSS-REFERENCE TO RELATED APPLICATION

This application claims priority to Japanese Patent Application No. 2015-158761 filed on Aug. 11, 2015. The entire disclosure of Japanese Patent Application No. 2015-158761 is hereby incorporated herein by reference.

### BACKGROUND

1. Technical Field

The present invention relates to a polymerase chain reaction device and a polymerase chain reaction method.

2. Related Art

As a method of selectively amplifying a target DNA (deoxyribonucleic acid) from a small amount of a genome (chromosome or gene) or an RNA (ribonucleic acid), there has been known a polymerase chain reaction (PCR) method developed by Dr. Kary Banks Mullis in the USA in 1983.

The PCR method is a method, which includes, for example, a thermal denaturation step of heating an aqueous solution containing a double-stranded DNA to be amplified, a primer which is a DNA fragment, a DNA synthesis material, and a DNA synthase to a given reaction temperature to dissociate the double-stranded DNA into single-stranded DNAs, an annealing step of binding the primer to the dissociated single-stranded DNA by cooling the aqueous solution from the given reaction temperature, and an elongation step of elongating the primer by further binding the DNA synthase to the primer bound to the single-stranded DNA, and exponentially amplifies the double-stranded DNA by sequentially repeating the three steps. PCR devices and PCR methods capable of performing such a polymerase chain reaction (PCR) have been developed.

For example, JP-A-2011-115159 (PTL 1) discloses a PCR device which includes a vessel for performing PCR, a pair of electrodes which are disposed facing each other across a gap along the flow of a reaction mixture on the inner surface of the vessel, and a control unit which controls the temperature of the reaction mixture by applying an alternating voltage to the pair of electrodes and allowing an alternating current to flow through the reaction mixture so as to generate Joule heat. It is described that according to this PCR device, an alternating current flows through the reaction mixture, and therefore, the reaction mixture is not electrolyzed, and also sufficient Joule heat for PCR cycles can be generated as compared with a method in which a reaction mixture is heated by allowing a direct current to flow through the reaction mixture to generate Joule heat.

Further, for example, JP-A-2011-188749 (PTL 2) discloses a DNA amplification device which includes a metal well in which a vessel (target substance) for performing PCR can be fitted, a temperature element which heats or cools the target substance by a Peltier effect, and a control section which controls the electrical conduction for the temperature element. Further, an example in which a p-type semiconductor and an n-type semiconductor are combined as the temperature element is shown. It is described that according to such a DNA amplification device, the time required for PCR can be reduced by allowing the temperature of the reaction mixture to follow the change in a given temperature pattern in PCR.

In the PCR device disclosed in the above PTL 1, an example in which a gap (channel) serving as a flow path through which a reaction mixture flows has a width of 980 μm, a depth of 600 μm, and a length of 2 to 8 mm is shown. In such a case, the volume of the reaction mixture filled in the gap comes to about 1 to 5 μL (microliters). An injection well for feeding the reaction mixture and a discharge well for discharging the reaction mixture are connected to the gap (channel), and therefore, in fact, it is necessary to prepare the reaction mixture in a larger amount than the volume of the gap (channel). In other words, the reaction mixture for performing PCR is likely to be wasted.

On the other hand, in the DNA amplification device disclosed in the above PTL 2, a reaction mixture is weighed and placed in a tube (vessel) in millimeters (mm), and the tube is placed in a metal well, and then, PCR is performed, and therefore, the wasteful consumption of the reaction mixture can be avoided. Then, it is described that the time required for one cycle of a temperature pattern at three levels in PCR can be reduced from about 250 seconds in the related art to 150 seconds. However, when the temperature pattern is repeated, for example, 50 cycles using the DNA amplification device disclosed in the above PTL 2, the time required for PCR comes to 7500 seconds, that is, about 2 hours, and therefore, there has been a demand for further reducing the time required for PCR.

### SUMMARY

An advantage of some aspects of the invention is to solve at least part of the problems described above and the invention can be implemented as the following aspects or application examples.

### Application Example

A polymerase chain reaction device according to this application example is a polymerase chain reaction device, with which a nucleic acid contained in a reaction mixture placed in a vessel is amplified, and includes a lower electrode and an upper electrode disposed spaced apart from each other in the vertical direction, an electric field generation section, and when the vessel is disposed between the lower electrode and the upper electrode, a first heating section which heats the vessel on a side near the upper electrode, a second heating section which heats the vessel on a side near the lower electrode, and a control section, wherein the vessel is filled with the reaction mixture and a liquid which has a lower specific gravity than the reaction mixture and is immiscible with the reaction mixture, and the control section drives and controls the first heating section and the second heating section so that the liquid in an upper part in the vessel is brought to a first temperature and the liquid in a lower part in the vessel is brought to a second temperature which is lower than the first temperature, and also drives and controls the electric field generation section to generate an electric field between the lower electrode and the upper electrode so that the reaction mixture in a spherical shape in the liquid moves up and down repeatedly between the upper part and the lower part of the liquid by a Coulomb force due to the electric field.

By using the polymerase chain reaction (PCR) device according to this application example, an electric field is generated between the lower electrode and the upper electrode to allow a Coulomb force to act on the reaction mixture in a spherical shape in the liquid, so that the reaction mixture is moved up to the upper part which is heated by the first heating section near the upper electrode in the liquid, and thus brought to the first temperature, and by stopping the generation of the electric field, the reaction mixture moved up is moved down to the lower part which is heated by the second heating section near the lower electrode in the liquid, and thus, the temperature of the reaction mixture can be quickly changed from the first temperature to the second temperature. By setting the first temperature to a temperature at which a nucleic acid is thermally denatured into single-stranded nucleic acids in the polymerase chain reaction (PCR), and the second temperature to an annealing or elongation temperature in PCR, which is lower than the first temperature, the temperature of the reaction mixture can be repeatedly changed in accordance with the temperature pattern for PCR. That is, it is possible to provide a PCR device capable of reducing the time required for nucleic acid amplification in PCR by reducing the time for switching the temperature pattern for PCR as compared with the PCR device of the related art disclosed in the above-mentioned PTL 1 or PTL 2 in which the temperature pattern for PCR is repeated by directly or indirectly heating the reaction mixture. In addition, since PCR is performed for the reaction mixture in the liquid which is immiscible with the reaction mixture, it is possible to omit the waste of the reaction mixture as compared with the PCR device disclosed in the above-mentioned PTL 1.

In the PCR device according to the above application example, it is preferred that the control section drives and controls the electric field generation section so that a first potential is applied to the lower electrode, and when the reaction mixture is positioned in the upper part, an alternating potential, in which the potential changes between the first potential and a second potential which is higher than the first potential, is applied to the upper electrode.

According to this configuration, a Coulomb force due to the alternating potential acts on the reaction mixture, and therefore, the reaction mixture minutely vibrates. That is, the reaction mixture is minutely stirred, so that the processes of thermal denaturation, annealing, and elongation can be enhanced. As a result, the time required for these processes can be reduced.

### Application Example

Another polymerase chain reaction device according to this application example is a polymerase chain reaction device, with which a nucleic acid contained in a reaction mixture placed in a vessel is amplified, and includes a lower electrode and an upper electrode disposed spaced apart from each other in the vertical direction, an electric field generation section, and when the vessel is disposed between the lower electrode and the upper electrode, a first heating section which heats the vessel on a side near the upper electrode, a second heating section which heats the vessel on a side near the lower electrode, and a control section, wherein the vessel is filled with the reaction mixture and a liquid which has a lower specific gravity than the reaction mixture and is immiscible with the reaction mixture, and the control section drives and controls the first heating section and the second heating section so that the liquid in an upper part in the vessel is brought to a first temperature and the liquid in a lower part in the vessel is brought to a second temperature which is lower than the first temperature, and also drives and controls the electric field generation section to generate an electric field between the lower electrode and the upper electrode so that the reaction mixture in a spherical shape in the liquid repeatedly moves up and down to parts in the following order: the upper part, the lower part, and a middle part to be brought to a third temperature between the upper part and the lower part of the liquid by a Coulomb force due to the electric field.

By using another PCR device according to this application example, an electric field is generated between the lower electrode and the upper electrode to allow a Coulomb force to act on the reaction mixture in a spherical shape in the liquid, so that the reaction mixture is moved up to the upper part which is heated by the first heating section near the upper electrode in the liquid, and thus brought to the first temperature, and by stopping the generation of the electric field, the reaction mixture moved up is moved down to the lower part which is heated by the second heating section near the lower electrode in the liquid, and thus, the temperature of the reaction mixture can be quickly changed from the first temperature to the second temperature. Further, by changing the intensity of the electric field, the magnitude of the Coulomb force can be adjusted so as to move the reaction mixture up to the middle part from the lower part, and thus, the temperature of the reaction mixture can be quickly changed from the second temperature to the third temperature. By setting the first temperature to a temperature at which a nucleic acid is thermally denatured into single-stranded nucleic acids in the polymerase chain reaction (PCR), the second temperature to an annealing temperature in PCR, which is lower than the first temperature, and the third temperature to an elongation temperature in PCR, the temperature of the reaction mixture can be repeatedly changed in accordance with the temperature pattern for PCR. That is, it is possible to provide a PCR device capable of reducing the time required for nucleic acid amplification in PCR by reducing the time for switching the temperature pattern for PCR as compared with the PCR device of the related art disclosed in the above-mentioned PTL 1 or PTL 2 in which the temperature pattern for PCR is repeated by directly or indirectly heating the reaction mixture. In addition, since PCR is performed for the reaction mixture in the liquid which is immiscible with the reaction mixture, it is possible to omit the waste of the reaction mixture as compared with the PCR device disclosed in the above-mentioned PTL 1.

In the PCR device according to the above application example, it is preferred that the control section drives and controls the electric field generation section so that a first potential is applied to the lower electrode, and when the reaction mixture is positioned in the upper part, an alternating potential, in which the potential changes between the first potential and a second potential which is higher than the first potential, is applied to the upper electrode, and when the reaction mixture is positioned in the middle part, an alternating potential, in which the potential changes between the first potential and a third potential which is lower than the second potential, is applied to the upper electrode.

According to this configuration, a Coulomb force due to the alternating potential acts on the reaction mixture, and therefore, the reaction mixture minutely vibrates. That is, the reaction mixture is minutely stirred, so that each of the processes of thermal denaturation, annealing, and elongation can be enhanced. As a result, the time required for these processes can be reduced.

In the PCR device according to the above application example, it is preferred that a stage capable of mounting a plurality of vessels thereon is included, and the lower electrode and the upper electrode are provided in common for the plurality of vessels.

According to this configuration, it is possible to provide a PCR device capable of simultaneously subjecting a lot of reaction mixtures of the same type or different types to PCR.

In the PCR device according to the above application example, it is preferred that the upper electrode is provided for each of the plurality of vessels, and has a columnar electrode section capable of being inserted into the vessel.

According to this configuration, the lower electrode and the columnar electrode section of the upper electrode are appropriately disposed for each of the plurality of vessels, and therefore, the Coulomb force can be effectively allowed to act on the reaction mixture placed in each of the plurality of vessels.

In the PCR device according to the above application example, it is preferred that the stage and the lower electrode are integrated with each other.

In the PCR device according to the above application example, it is preferred that the lower electrode and the second heating section are integrated with each other.

According to these configurations, it is possible to provide a PCR device having a simple structure by reducing the number of components.

In the PCR device according to the above application example, it is preferred that a lifting mechanism capable of adjusting an interelectrode distance between the lower electrode and the upper electrode by moving at least one of the lower electrode and the upper electrode is included.

According to this configuration, the interelectrode distance between the lower electrode and the upper electrode can be adjusted by the lifting mechanism, and therefore, the Coulomb force can be effectively allowed to act on the reaction mixture. In other words, the Coulomb force to move the reaction mixture is determined mainly by the potential to be applied to the lower electrode and the upper electrode and the interelectrode distance, and therefore, the wasteful power consumption can be reduced by adjusting the interelectrode distance to be appropriate in accordance with the potential to be applied.

### Application Example

A polymerase chain reaction (PCR) method according to this application example is a polymerase chain reaction method, with which a nucleic acid contained in a reaction mixture is amplified, and includes a first step of filling a vessel with the reaction mixture and a liquid which has a lower specific gravity than the reaction mixture and is immiscible with the reaction mixture, a second step of heating an upper part of the liquid filled in the vessel to a first temperature at which the nucleic acid is thermally denatured, and also heating a lower part of the liquid filled in the vessel to a second temperature, which is lower than the first temperature, and at which the thermally denatured nucleic acid is amplified, and a third step of moving the reaction mixture up and down repeatedly between the upper part and the lower part of the liquid by generating an electric field between the lower electrode and the upper electrode disposed spaced apart from each other in the vertical direction with respect to the vessel and allowing a Coulomb force to act on the reaction mixture in a spherical shape in the liquid.

By using the PCR method according to this application example, an electric field is generated between the lower electrode and the upper electrode to allow a Coulomb force to act on the reaction mixture in a spherical shape in the liquid, so that the reaction mixture is moved up and positioned in the upper part in the liquid, and thus brought to the first temperature, and by stopping the generation of the electric field, the reaction mixture having the first temperature and positioned in the upper part is moved down and positioned in the lower part in the liquid, and thus, the temperature of the reaction mixture can be quickly changed to the second temperature. That is, the temperature of the reaction mixture can be repeatedly changed between the first temperature and the second temperature in accordance with the temperature pattern for PCR. Accordingly, it is possible to provide a PCR method capable of reducing the time required for nucleic acid amplification in PCR by reducing the time for switching the temperature pattern for PCR as compared with a PCR method of the related art using the PCR device disclosed in the above-mentioned PTL 1 or PTL 2 in which the temperature pattern for PCR is repeated by directly or indirectly heating the reaction mixture. In addition, since PCR is performed for the reaction mixture in the liquid which is immiscible with the reaction mixture, it is possible to omit the waste of the reaction mixture as compared with the PCR method of the related art using the PCR device disclosed in the above-mentioned PTL 1.

In the PCR method according to the above application example, it is preferred that a first potential is applied to the lower electrode, and when the reaction mixture is positioned in the upper part, an alternating potential, in which the potential changes between the first potential and a second potential which is higher than the first potential, is applied to the upper electrode.

According to this method, a Coulomb force due to the alternating potential acts on the reaction mixture, and therefore, the reaction mixture minutely vibrates. That is, the reaction mixture is minutely stirred, so that the reactions in the second step and the third step can be enhanced. As a result, the time required for these steps can be reduced.

In the PCR method according to the above application example, it is preferred that the reaction mixture contains a target nucleic acid, a nucleic acid synthesis substrate, a heat-resistant enzyme, and a primer, and the third step includes a fourth step of thermally denaturing and separating the target nucleic acid into single-stranded nucleic acids at the first temperature, a fifth step of binding the primer to the single-stranded nucleic acid at the second temperature, and a sixth step of synthesizing a nucleic acid complementary to a single-stranded portion at the second temperature using the heat-resistant enzyme as the catalyst and also using the nucleic acid synthesis substrate with the primer bound to the single-stranded nucleic acid as the origin.

According to this method, the target nucleic acid can be amplified by efficiently repeating the steps from the fourth step to the sixth step.

In the PCR method according to the above application example, it is preferred that the liquid in the vessel has a middle part brought to a third temperature which is lower than the first temperature and higher than the second temperature between the upper part heated to the first temperature and the lower part heated to the second temperature, the reaction mixture contains a target nucleic acid, a nucleic acid synthesis substrate, a heat-resistant enzyme, and a primer, the third step includes a fourth step of thermally denaturing and separating the target nucleic acid into single-stranded nucleic acids at the first temperature, a fifth step of binding the primer to the single-stranded nucleic acid at the second temperature, and a sixth step of synthesizing a nucleic acid complementary to a single-stranded portion at the third temperature using the heat-resistant enzyme as the catalyst and also using the nucleic acid synthesis substrate with the primer bound to the single-stranded nucleic acid as the origin, and the reaction mixture is repeatedly moved up and down to parts in the following order: the upper part, the lower part, and the middle part of the liquid by generating an electric field between the lower electrode and the upper electrode and allowing a Coulomb force to act on the reaction mixture in a spherical shape in the liquid.

According to this method, the reaction can be performed by setting the temperatures in the fifth step and the sixth step to appropriate temperatures, respectively, and therefore, the target nucleic acid can be amplified by more efficiently repeating the steps from the fourth step to the sixth step.

In the PCR method according to the above application example, it is preferred that a first potential is applied to the lower electrode, and when the reaction mixture is positioned in the upper part, an alternating potential, in which the potential changes between the first potential and a second potential which is higher than the first potential, is applied to the upper electrode, and when the reaction mixture is positioned in the middle part, an alternating potential, in which the potential changes between the first potential and a third potential which is lower than the second potential, is applied to the upper electrode.

According to this method, a Coulomb force due to the alternating potential acts on the reaction mixture, and therefore, the reaction mixture minutely vibrates. That is, the reaction mixture is minutely stirred, so that the reactions in the second step and the third step can be enhanced. As a result, the time required for these steps can be reduced.

In the PCR method according to the above application example, it is preferred that the target nucleic acid is a DNA.

According to this method, a PCR method capable of efficiently amplifying a DNA can be provided.

In the PCR method according to the above application example, it is preferred that the target nucleic acid is a nucleic acid in which two single-stranded RNAs are bound to each other.

According to this method, a PCR method capable of efficiently amplifying a single-stranded RNA can be provided.

### BRIEF DESCRIPTION OF THE DRAWINGS

The invention will be described with reference to the accompanying drawings, wherein like numbers reference like elements.

FIG. 1 is a schematic view showing an example of an electrical and mechanical structure of a PCR device according to a first embodiment.

FIG. 2 is a schematic perspective view showing a vessel.

FIG. 3 is a schematic plan view showing a heating section.

FIG. 4A is a schematic view showing an operation of the PCR device according to the first embodiment.

FIG. 4B is a schematic view showing an operation of the PCR device according to the first embodiment.

FIG. 5A is a view showing a waveform of an alternating potential to be applied to an upper electrode.

FIG. 5B is a graph showing one example of a change in the temperature of a reaction mixture.

FIG. 6 is a schematic view showing each step of a PCR method.

FIG. 7 is a schematic view showing a thermal denaturation reaction of a DNA.

FIG. 8 is a schematic view showing an annealing reaction.

FIG. 9 is a schematic view showing an elongation reaction.

FIG. 10 is a schematic view showing a PCR device according to a second embodiment.

FIG. 11 is a schematic perspective view showing a vessel to be used in the PCR device according to the second embodiment.

FIG. 12 is a schematic plan view showing a heating section to be used in the PCR device according to the second embodiment.

FIG. 13 is a schematic view showing steps of another PCR method.

FIG. 14A is a view showing a waveform of an alternating potential to be applied to an upper electrode in another PCR method.

FIG. 14B is a graph showing one example of a change in the temperature of a reaction mixture in another PCR method.

### DESCRIPTION OF EXEMPLARY EMBODIMENTS

Hereinafter, embodiments embodying the invention will be described with reference to the drawings. The drawings to be used are displayed by appropriately enlarging or reducing the size so as to make portions to be described recognizable.

### First Embodiment

### Polymerase Chain Reaction (PCR) Device

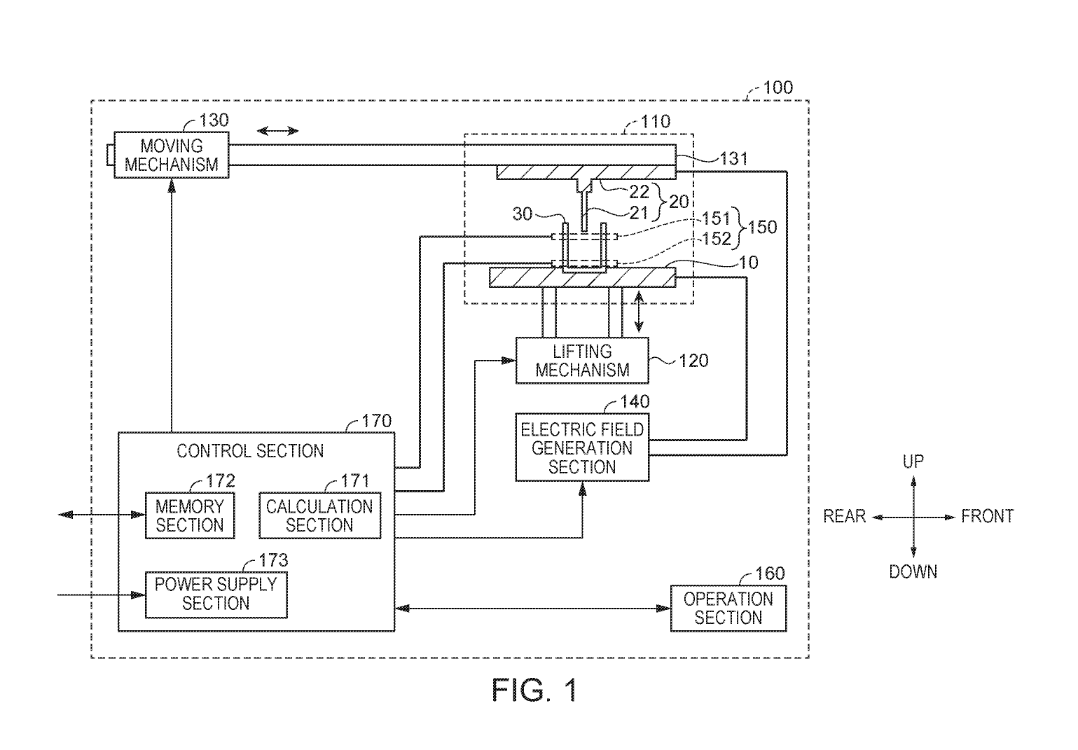

An example of the entire structure of a polymerase chain reaction (PCR) device to be used for a polymerase chain reaction (PCR) according to this embodiment will be described with reference to FIG. 1. FIG. 1 is a schematic view showing an example of an electrical and mechanical structure of the PCR device. The PCR device according to this embodiment is a device, with which a nucleic acid contained in a reaction mixture is amplified by repeatedly performing a temperature pattern (heating and cooling) in PCR for a reaction mixture placed in a vessel.

As shown in FIG. 1, a PCR device 100 of this embodiment is configured to include a lower electrode 10, an upper electrode 20, a lifting mechanism 120, a moving mechanism 130, an electric field generation section 140, a heating section 150, an operation section 160, and a control section 170. The lower electrode 10 and the upper electrode 20 can be disposed facing each other in the vertical direction, and a vessel 30 in which a reaction mixture is placed is mounted on the surface of the lower electrode 10. A space in which the lower electrode 10 and the upper electrode 20 are disposed facing each other is a treatment chamber 110. The treatment chamber 110 is composed of a wall (not shown) which separates the chamber from the surrounding space, an openable and closable lid (not shown), and the like, and the treatment chamber 110 can be brought to a substantially closed state and an open state. The PCR device 100 includes a housing (not shown) in which the treatment chamber 110, the lifting mechanism 120, the moving mechanism 130, the electric field generation section 140, the heating section 150, the operation section 160, and the control section 170 are provided.

When the treatment chamber 110 is used as the base, the lifting mechanism 120 and the electric field generation section 140 are provided on the lower side of the treatment chamber 110. The operation section 160 is provided on the front side of the treatment chamber 110. The moving mechanism 130 and the control section 170 are provided on the rear side of the treatment chamber 110. Hereinafter, in the drawing, the vertical direction in which the lower electrode 10 and the upper electrode 20 are disposed facing each other is referred to as “up-and-down direction”, and the front and rear direction orthogonal to the up-and-down direction is referred to as “front-and-rear direction”. Further, although not shown in FIG. 1, the description will be made by referring to the right and left direction orthogonal to the up-and-down direction as “right-and-left direction”.

The lower electrode 10 is composed of, for example, an aluminum plate subjected to an alumite treatment, and has a mounting section capable of mounting the vessel 30 in a predetermined position on the surface on the upper side in the up-and-down direction. Therefore, the lower electrode 10 functions as a stage on which the vessel 30 is mounted. In other words, the stage and the lower electrode 10 are integrated with each other.

The upper electrode 20 includes a columnar electrode section 21 and an electrode support section 22. The electrode support section 22 is composed of, for example, an aluminum plate subjected to an alumite treatment. The columnar electrode section 21 is composed of, for example, an aluminum rod subjected to an alumite treatment, and is erected on the surface on the lower side in the up-and-down direction of the electrode support section 22.

The lifting mechanism 120 can move the lower electrode 10 up and down. According to this, when the lower electrode 10 and the upper electrode 20 are in a state of being disposed facing each other in the up-and-down direction, the interelectrode distance between the lower electrode 10 and the upper electrode 20 (practically, the tip of the columnar electrode section 21) can be adjusted.

The moving mechanism 130 can move a support section 131, which extends in the front-and-rear direction, in the front-and-rear direction. The upper electrode 20 (electrode support section 22) is attached to the lower surface on the front side of the support section 131.

The electric field generation section 140 generates an electric field between the lower electrode 10 and the upper electrode 20 (actually, the columnar electrode section 21) by applying a potential to each of the lower electrode 10 and the upper electrode 20. Specifically, as the first potential, for example, 0 V is applied to the lower electrode 10, and as the second potential, for example, an alternating potential, in which the potential changes between 0 V and 6 kV, is applied to the upper electrode 20. A detailed method of applying such an alternating potential will be described later, however, the alternating potential is periodically applied. In this embodiment, a configuration in which the lower electrode 10, the upper electrode 20, and the electric field generation section 140 are provided independently is adopted, however, a configuration in which the lower electrode 10 and the upper electrode 20 are included in the electric field generation section 140 may be adopted.

The heating section 150 includes a first heating section 151 capable of heating an upper part of the vessel 30 mounted on the lower electrode 10 and a second heating section 152 capable of heating a lower part of the vessel 30.

The operation section 160 includes, for example, a display section such as a liquid crystal display panel and an input section of a touch panel system superimposed on the display section, and various operation buttons displayed on the display section can be selected by the input section.

Each of the lifting mechanism 120, the moving mechanism 130, the electric field generation section 140, the heating section 150, and the operation section 160 is electrically connected to the control section 170. According to this, the control section 170 drives and controls each section of the PCR device 100 to perform an operation corresponding to the above-mentioned various operation buttons. For example, the control section 170 drives and controls the lifting mechanism 120 to move the lower electrode 10 in the up-and-down direction based on the operation from the operation section 160, and thus, the interelectrode distance between the lower electrode 10 and the upper electrode 20 disposed facing each other can be adjusted. Further, for example, the control section 170 drives and controls the moving mechanism 130 to move the support section 131 in the front-and-rear direction, and thus, the upper electrode 20 attached on the front side of the support section 131 can be moved between a first position facing the lower electrode 10 and a second position retracted from the first position. Further, for example, the control section 170 drives and controls the electric field generation section 140 to apply a potential to each of the lower electrode 10 and the upper electrode 20, and thus, an electric field can be periodically generated between the lower electrode 10 and the upper electrode 20. Further, for example, the control section 170 drives and controls the heating section 150 to heat the vessel 30 by the first heating section 151 and the second heating section 152, and thus, the upper part of the vessel 30 can be brought to a first temperature, and the lower part of the vessel 30 can be brought to a second temperature which is lower than the first temperature.

The control section 170 includes a calculation section 171, a memory section 172, and a power supply section 173, and can automatically drive and control each of the above-mentioned sections by supplying power to each section from the power supply section 173 based on a PCR program previously stored in the memory section 172. The PCR program includes information as to the reaction conditions for causing various reactions in PCR. In the memory section 172, not only the above-mentioned PCR program, but also various control programs of the PCR device 100 are included, and the PCR program can be viewed, corrected, or added by accessing the memory section 172 from the outside through an interface provided in the control section 170. As for the access from the outside, from the viewpoint of ensuring security, it is also possible to set an access code or the like. Further, the power supply section 173 is not limited to the configuration in which the power supply section 173 is included in the control section 170, and may be configured to be provided independently and electrically controlled by the control section 170.

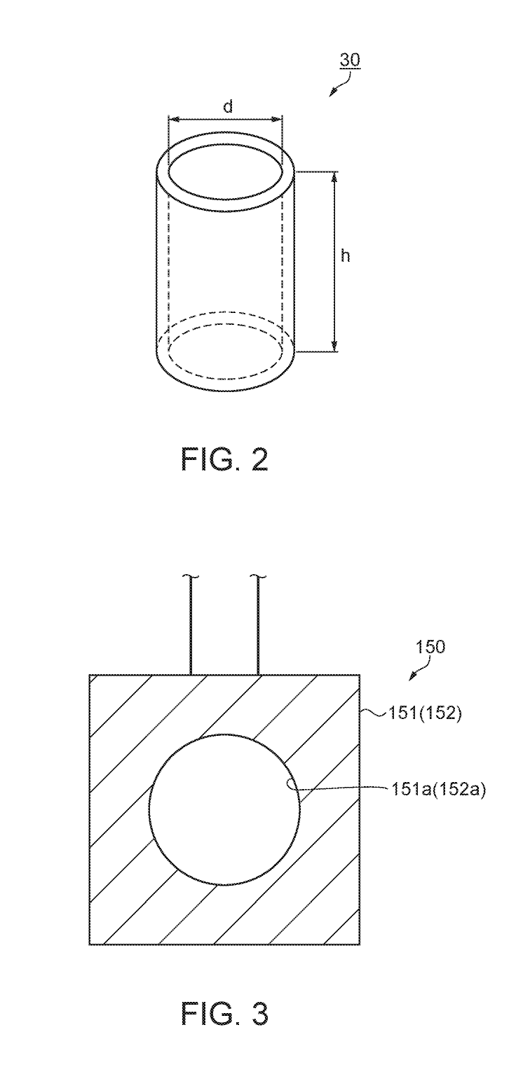

Next, the vessel 30 will be described with reference to FIG. 2. FIG. 2 is a schematic perspective view showing the vessel. As shown in FIG. 2, the vessel 30 is a cylindrical vessel with a flat bottom formed using a light transmitting material. As the light transmitting material, a glass, a plastic, or the like can be used. When the volume of the vessel 30 is, for example, about 400 μL and the height h of the inner part of the vessel 30 is, for example, 12 mm, the inner diameter d of the vessel 30 is about 6.5 mm. As shown in FIG. 1, the columnar electrode section 21 of the upper electrode 20 can be inserted into the vessel 30. Therefore, the thickness of the columnar electrode section 21 is smaller than the inner diameter d of the vessel 30, and is, for example, from about 1 mm to 3 mm. The length of the columnar electrode section 21 is, for example, from about 10 mm to 20 mm.

Next, the heating section 150 will be described with reference to FIG. 3. FIG. 3 is a schematic plan view showing the heating section. Specifically, FIG. 3 shows the first heating section 151 of the heating section 150.

As shown in FIG. 3, the first heating section 151 of the heating section 150 is, for example, a ceramic heater in a flat plate shape with a hole 151a in the center. The hole 151a has a size such that a small gap is formed between the inner wall of the hole 151a and the outer wall of the vessel 30 so that the vessel 30 can be smoothly inserted into the hole 151a. The second heating section 152 of the heating section 150 has the same structure as that of the first heating section 151 and has a hole 152a into which the vessel 30 can be inserted in the same manner.

The second heating section 152 disposed so as to be able to heat the lower part of the vessel 30 may be integrally formed with the lower electrode 10. According to this, by inserting the vessel 30 into the hole 152a of the second heating section 152, the vessel 30 can be mounted by determining the position with respect to the lower electrode 10, and thus, the lower electrode 10 can be configured to be able to exhibit the function of the stage. After the vessel 30 is inserted into the hole 152a of the second heating section 152, the first heating section 151 is placed from above the vessel 30. The first heating section 151 is placed spaced apart at a predetermined distance from the second heating section 152. A detailed positional relationship between the first heating section 151 and the second heating section 152 in the up-and-down direction will be described later.

### Operation of PCR Device

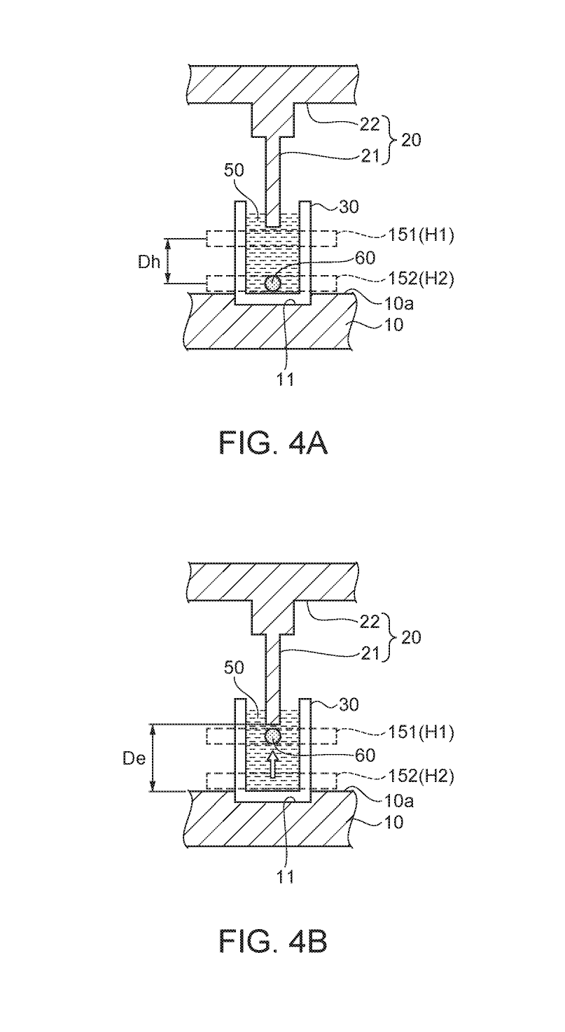

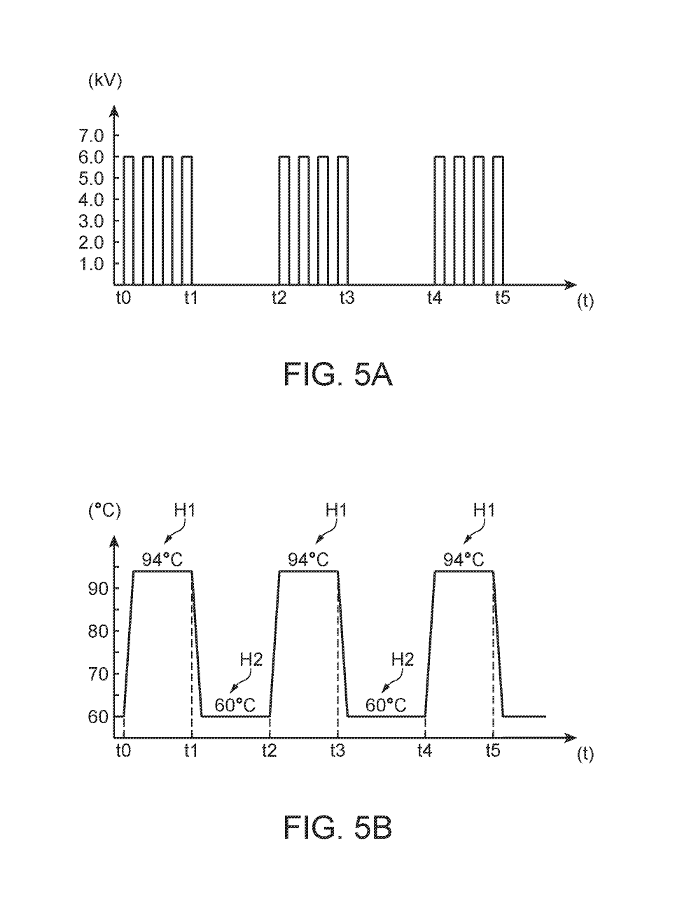

Next, an operation of the PCR device 100 of this embodiment will be described with reference to FIGS. 4A, 4B, 5A, and 5B. FIGS. 4A and 4B are schematic views showing an operation of the PCR device, FIG. 5A is a view showing a waveform of an alternating potential to be applied to the upper electrode, and FIG. 5B is a graph showing one example of a change in the temperature of a reaction mixture.

As shown in FIG. 4A, in the vessel 30, a liquid 50 and a reaction mixture 60 are placed. The reaction mixture 60 is basically an aqueous solution and has a specific gravity higher than “1”. On the other hand, as the liquid 50, a substance which has a lower specific gravity than the reaction mixture 60 and is immiscible with the reaction mixture 60 is selected. The amount (volume) of the reaction mixture 60 to be placed in the vessel 30 is smaller than the amount (volume) of the liquid 50, and for example, 10 μL or less. Therefore, in the liquid 50, the reaction mixture 60 has a spherical shape due to the interfacial tension between the reaction mixture 60 and the liquid 50. Further, the reaction mixture 60 has a higher specific gravity than the liquid 50, and therefore sinks to the bottom of the vessel 30.

As described above, the vessel 30 is composed of a material with a light transmitting property, and the liquid 50 to be placed therein is also a substance with a light transmitting property. Examples of the substance constituting such a liquid 50 include a silicone-based oil. The “light transmitting property” in this embodiment refers to a state with a transmittance of 80% or more in a visible light wavelength region.

The vessel 30 in which the liquid 50 and the reaction mixture 60 are placed is mounted on the lower electrode 10. On the surface 10a of the lower electrode 10, amounting section 11 for mounting the vessel 30 in a predetermined position is provided. In this embodiment, a concave portion corresponding to the size of the bottom surface of the vessel 30 is provided on the surface 10a of the lower electrode 10 and is used as the mounting section 11. The depth of the concave portion corresponds to the thickness of the bottom of the vessel 30. The configuration of the mounting section 11 is not limited thereto, and a convex portion which determines the position of the vessel 30 may be provided on the surface 10a of the lower electrode 10.

With respect to the vessel 30 mounted on the lower electrode 10, the second heating section 152 is disposed on a side near the lower electrode 10. Further, the first heating section 151 is disposed above and spaced apart at a predetermined distance Dh from the disposed second heating section 152. The predetermined distance Dh is, for example, from 5 mm to 10 mm. In this embodiment, a region to be heated by the first heating section 151 with respect to the vessel 30 is called “first region H1”, and similarly, a region to be heated by the second heating section 152 with respect to the vessel 30 is called “second region H2”. The first region H1 corresponds to the upper part to be brought to the first temperature in the invention, and the second region H2 corresponds to the lower part to be brought to the second temperature in the invention. When the reaction mixture 60 in a spherical shape in the liquid 50 sinks, the reaction mixture 60 is positioned in the second region H2 with respect to the vessel 30.

The control section 170 drives and controls the moving mechanism 130 to move the upper electrode 20 so that the columnar electrode section 21 is positioned above the vessel 30. Subsequently, the control section 170 drives and controls the lifting mechanism 120 to lift the lower electrode 10 so that the columnar electrode section 21 is inserted into the vessel 30 in which the liquid 50 and the reaction mixture 60 are placed. Further, as shown in FIG. 4B, the interelectrode distance between the lower electrode 10 and the upper electrode 20 (practically, the tip of the columnar electrode section 21) is adjusted to be a desired distance De. Hereinafter, the desired distance De is called “interelectrode distance De”. As described above, the mounting section 11 is provided in the lower electrode 10, and therefore, the practical interelectrode distance De is adjusted in consideration of the depth of the mounting section 11.

Further, the control section 170 drives and controls the electric field generation section 140 to apply a potential to the lower electrode 10 and the upper electrode 20 disposed at the interelectrode distance De, thereby generating an electric field. Specifically, for example, as shown in FIG. 5A, the potential of the lower electrode 10 is set to 0 V, and for example, an alternating potential at a frequency of 30 Hz in which the potential changes between 0 V and 6 kV is applied to the upper electrode 20 at a period based on the PCR program. Incidentally, in FIG. 5A, the waveform of the alternating potential is shown to such an extent that the application state of the alternating potential can be distinguished, and therefore is different from the waveform of the alternating potential to be actually applied. Further, the waveform of the alternating potential may be a rectangular pulse waveform (digital waveform) as shown in FIG. 5A or may be a sine waveform (analog waveform) in which the potential continuously changes.

In a period, for example, from a time t₀ to a time t₁ in which the alternating potential is applied to the upper electrode 20, an electric field is generated between the lower electrode 10 and the columnar electrode section 21 of the upper electrode 20, and a Coulomb force due to the electric field acts on the react ion mixture 60 in a spherical shape. Therefore, as shown in FIG. 4B, the reaction mixture 60 in a spherical shape is attracted toward the columnar electrode section 21 in the liquid 50 and moves up. The upward movement of the reaction mixture 60 is stopped at a position where the Coulomb force which acts on the reaction mixture 60 and the force of gravity are balanced with each other. In fact, an alternating potential is applied to the upper electrode 20, and therefore, the reaction mixture 60 on which the Coulomb force acts minutely vibrates in accordance with the frequency of the alternating potential.

In a period, for example, from a time t₁ to a time t₂ in which an alternating potential is not applied to the upper electrode 20, the electric field between the lower electrode 10 and the columnar electrode section 21 of the upper electrode 20 is lost, and therefore, a Coulomb force does not act on the reaction mixture 60 in a spherical shape so that the reaction mixture 60 moves down in the liquid 50 due to the force of gravity. As a result, the reaction mixture 60 is positioned in the second region H2 as shown in FIG. 4A.

The alternating potential to be applied to the upper electrode 20 is not limited to an alternating potential from 0 V to 6 kV at a frequency of 30 Hz. The voltage and frequency of the alternating potential, and the interelectrode distance De are set in consideration of the specific gravity and mass of the reaction mixture 60, the specific gravity and viscosity of the liquid 50, and the like so that the reaction mixture 60 moves up in the liquid 50 and is positioned in the first region H1 by applying the alternating potential to the upper electrode 20. For example, in the case where the specific gravity of the reaction mixture 60 is set to substantially 1.0, and the volume thereof is set to 10 μL (microliters) or less, and a silicone-based oil having a specific gravity of about 0.89 is used as the liquid 50, an alternating potential in the range of 0 V to 10 kV at a frequency in the range of 0.2 Hz to 50 Hz is conceivable.

The first temperature of the liquid 50 in the first region H1 to be heated by the first heating section 151 is set to, for example, 94° C., and the second temperature of the liquid 50 in the second region H2 to be heated by the second heating section 152 is set to, for example, 60° C. By doing this, as shown in FIG. 5B, the temperature of the reaction mixture 60 which moves up in the liquid 50 and is positioned in the first region H1 by applying an alternating potential to the upper electrode 20 reaches 94° C. and maintained until the time t₁. The temperature of the reaction mixture 60 which moves down in the liquid 50 and is positioned in the second region H2 by stopping the application of the alternating potential to the upper electrode 20 is decreased (cooled) from 94° C. to 60° C. and maintained until the time t₂.

When the potential of the lower electrode 10 is set to 0 V and an alternating potential is periodically applied to the upper electrode 20, the reaction mixture 60 in a spherical shape in the liquid 50 periodically moves up and down between the second region H2 and the first region H1. That is, the temperature of the reaction mixture 60 periodically changes between 94° C. and 60° C. as shown in FIG. 5B. The operation of such a PCR device 100 can quickly change the temperature of the reaction mixture 60 as compared with the PCR device of the related art in which the temperature is changed between the first temperature and the second temperature by directly or indirectly heating and cooling the reaction mixture which is left to stand.

In consideration of the adjustment of the intensity of the electric field to be generated between the lower electrode 10 and the upper electrode 20, it is preferred that the state where the reaction mixture 60 in a spherical shape moves up and down in the liquid 50 can be visually observed. Therefore, it is preferred that the vessel 30 and the liquid 50 have a light transmitting property.

### Polymerase Chain Reaction (PCR) Method

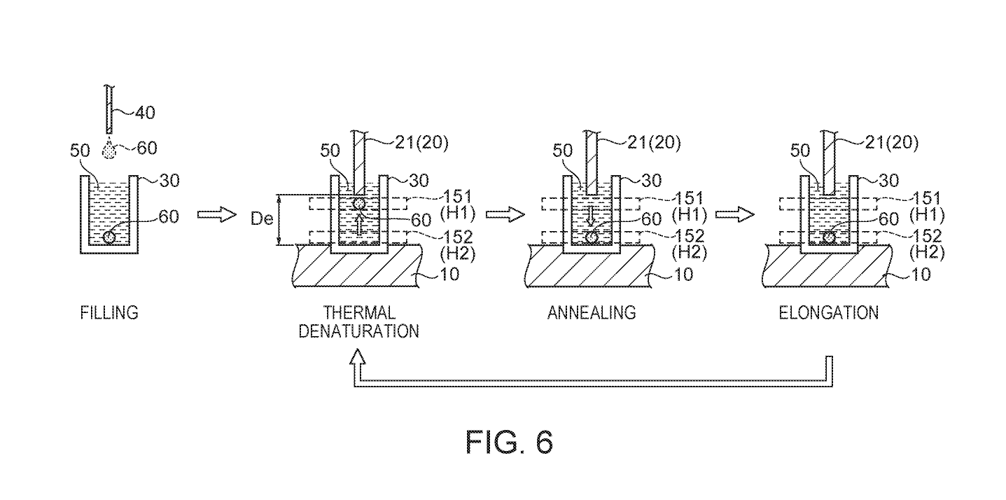

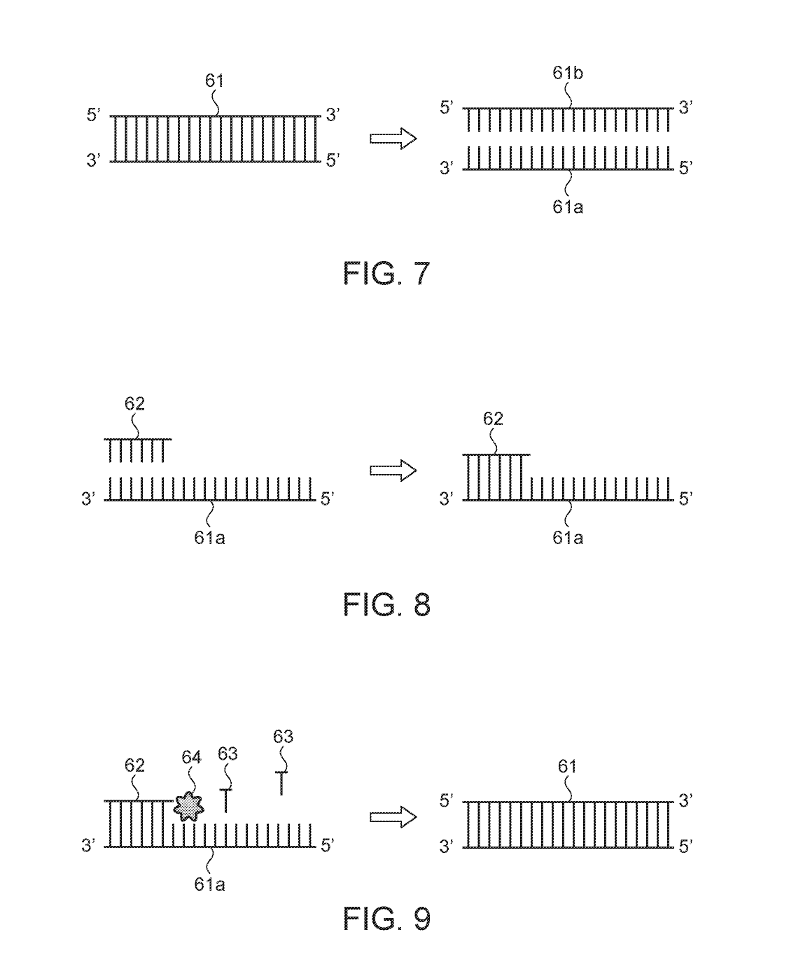

Next, the PCR method according to this embodiment will be described with reference to FIGS. 6 to 9. FIG. 6 is a schematic view showing each step of the PCR method, FIG. 7 is a schematic view showing a thermal denaturation reaction of a DNA, FIG. 8 is a schematic view showing an annealing reaction, and FIG. 9 is a schematic view showing an elongation reaction.

The PCR method according to this embodiment is a method with which a DNA as a target nucleic acid contained in the reaction mixture 60 is amplified, and includes a filling step (first step) of filling the vessel 30 with the liquid 50 and the reaction mixture 60, a heating step (second step), a thermal denaturation step (fourth step), an annealing step (fifth step), an elongation step (sixth step), and a step of repeating these steps in this order (third step).

In the reaction mixture 60, a DNA as a target nucleic acid, a DNA synthesis substrate (dNTP: deoxynucleotide triphosphate), a heat-resistant enzyme (heat-resistant DNA polymerase), a primer (oligonucleotide), and water are contained. Incidentally, in the reaction mixture 60, at least two types of primers called a forward primer and a reverse primer are contained.

Specifically, as shown in FIG. 6, in the filling step, after the vessel 30 is filled with the liquid 50, a predetermined amount (for example, 5 μL) of the reaction mixture 60 containing a DNA as a target nucleic acid is discharged into the vessel 30 from, for example, a micropipette 40 capable of discharging a fixed amount of a liquid in a small amount. A liquid droplet of the discharged reaction mixture 60 has a higher specific gravity than the liquid 50 and is immiscible with the liquid 50, and therefore is in a spherical shape in the liquid 50 and sinks to the bottom of the vessel 30. The vessel 30 in which the liquid 50 and the reaction mixture 60 are placed is disposed on the lower electrode 10 of the PCR device 100. Then, the process proceeds to the heating step.

In the heating step, the PCR device 100 is operated, and the control section 170 drives and controls the first heating section 151 and the second heating section 152, whereby the temperature of the liquid 50 in the first region H1 in the vessel 30 is brought to the first temperature, and also the temperature of the liquid 50 in the second region H2 in the vessel 30 is brought to the second temperature. Then, the process proceeds to the third step.

In the third step, the control section 170 drives and controls the moving mechanism 130 to move the upper electrode 20 so that the columnar electrode section 21 is positioned above the vessel 30. Then, the control section 170 drives and controls the lifting mechanism 120 to lift the lower electrode 10 so that the interelectrode distance becomes the desired interelectrode distance De. By doing this, the columnar electrode section 21 is inserted into the vessel 30, and the tip of the columnar electrode section 21 is dipped in the liquid 50 and is stopped at a position slightly above the first region H1. In this state, the control section 170 drives and controls the electric field generation section 140 to periodically generate an electric field between the lower electrode 10 and the upper electrode 20.

In a period in which an alternating potential is applied to the upper electrode 20, a Coulomb force due to the electric field generated between the lower electrode 10 and the columnar electrode section 21 acts on the reaction mixture 60, so that the reaction mixture 60 in a spherical shape is attracted to the columnar electrode section 21 and moves up, and then stops in the first region H1 and is brought to a minutely vibrating state. The temperature of the reaction mixture 60 in the first region H1 is increased to, for example, 94° C. and maintained as shown in FIG. 5B. As shown in FIG. 7, a double-stranded DNA 61 contained in the reaction mixture 60 is separated into two single-stranded DNAs 61a and 61b by heating the reaction mixture 60 to 94° C. The process up to this point is the thermal denaturation step (fourth step) in the third step. Incidentally, the temperature of the liquid 50, that is, the temperature of the reaction mixture 60 in the thermal denaturation step is set to a temperature equal to or higher than 95° C. and lower than 100° C., at which the double-stranded DNA 61 is separated and also the reaction mixture 60 does not boil. The sign “3′” or “5′” shown in the ends of the double-stranded DNA 61 and the single-stranded DNAs 61a and 61b indicates the position of carbon of a sugar in a nucleotide which is a constituent unit. A nucleic acid forms a chain structure in which the carbon at the 3′-position and the carbon at the 5′-position of a sugar are bound to phosphoric acid through a phosphoester bond, and an end at which the 5′-phosphoester bond is cleaved is referred to as and the other end is referred to as “3′-end”. The signs “3′” and “5′” shown here indicate “3′-end” and “5′-end”, respectively. Then, the process proceeds to the annealing step (fifth step).

In the annealing step (fifth step), the application of the alternating potential to the upper electrode 20 is stopped. By doing this, the electric field generated between the lower electrode 10 and the upper electrode 20 is lost, and the Coulomb force no longer acts on the reaction mixture 60. Therefore, the reaction mixture 60 in a spherical shape moves down to the second region H2 from the first region H1 due to the force of gravity. In a period in which an alternating potential is not applied to the upper electrode 20, the temperature of the reaction mixture 60 in the second region H2 is decreased (cooled), for example, from 94° C. to 60° C. as shown in FIG. 5B, and maintained. As shown in FIG. 8, in the reaction mixture 60, a primer 62 binds to the single-stranded DNA 61a. The primer 62 binds to a portion having a complementary base sequence of the single-stranded DNA 61a. Then, the process proceeds to the elongation step (sixth step).

In the elongation step (sixth step), a reaction proceeds in a state where an alternating potential is not applied to the upper electrode 20, that is, in a state where the reaction mixture 60 in a spherical shape is retained in the second region H2 and is brought to the second temperature (60° C.). Specifically, as shown in FIG. 9, a DNA synthesis substrate 63 is sequentially bound using a heat-resistant enzyme 64 as the catalyst with the primer 62 bound to the single-stranded DNA 61a as the origin to synthesize a complementary strand to the single-stranded DNA 61a, whereby the double-stranded DNA 61 is formed. Also for the other single-stranded DNA 61b separated in the thermal denaturation step, the annealing reaction and the elongation reaction proceed at the second temperature (60° C.), and a complementary strand to the single-stranded DNA 61b is synthesized, and thus, the double-stranded DNA 61 is formed.

By repeatedly performing the above-mentioned steps from the thermal denaturation step to the elongation step n times, the double-stranded DNA 61 is amplified by 2ⁿ times. In the PCR method, by repeating such steps from the thermal denaturation step to the elongation step, the DNA synthesis substrate 63 is consumed, and the concentration of the DNA synthesis substrate 63 decreases. In general, the cycle from the thermal denaturation step to the elongation step is repeated about 50 times.

Examples of a method of confirming that the amplification of the target nucleic acid has occurred include a method in which a fluorescent probe is added to the reaction mixture 60. It can be examined whether or not the amplification of the target nucleic acid has occurred by measuring the fluorescence emitted from a fluorescent substance contained in the fluorescent probe.

According to the above-mentioned PCR device 100 of the first embodiment and the PCR method using this device, the following effects can be obtained.

(1) By utilizing a Coulomb force due to an electric field generated between the lower electrode 10 and the columnar electrode section 21, in the liquid 50 filled in the vessel 30, the reaction mixture 60 is moved up and down between the first region H1 (upper part) to be brought to the first temperature at which a thermal denaturation reaction in PCR proceeds and the second region H2 (lower part) to be brought to the second temperature, which is lower than the first temperature, and at which an annealing reaction and an elongation reaction in PCR proceed. Therefore, as compared with the PCR device of the related art in which the reaction mixture 60 which is left to stand is directly or indirectly heated and cooled, and the PCR method using this device, the temperature of the reaction mixture 60 can be changed in a shorter time, and therefore, the PCR device 100 capable of reducing the time required for PCR and a PCR method using this device can be provided.

(2) When a Coulomb force due to an electric field is generated, a first potential (0 V) is applied to the lower electrode 10 and an alternating potential, in which the potential changes between the first potential and a second potential (6 kV) which is higher than the first potential (0 V), is applied to the upper electrode 20. By doing this, minute vibration corresponding to the frequency of the alternating potential occurs in the reaction mixture 60, and the reaction mixture 60 is minutely stirred. By minutely stirring the reaction mixture 60, the thermal denaturation reaction, the annealing reaction, and the elongation reaction efficiently proceed, and therefore, the time required for PCR can be further reduced.

(3) As the liquid 50 filled in the vessel 30, a material which has a lower specific gravity than the reaction mixture 60 which is an aqueous solution and is immiscible with the reaction mixture 60 is selected, and therefore, the reaction mixture 60 is in a spherical shape in the liquid 50. In the PCR device 100 and the PCR method using this device, the respective reactions in PCR are performed for the reaction mixture 60 in a spherical shape, and therefore, as compared with the PCR in the related art in which the liquid 50 is not used, PCR can be performed even for the reaction mixture 60 in a small amount. In other words, it is possible to omit the waste of the reagent constituting the reaction mixture 60.

### Second Embodiment

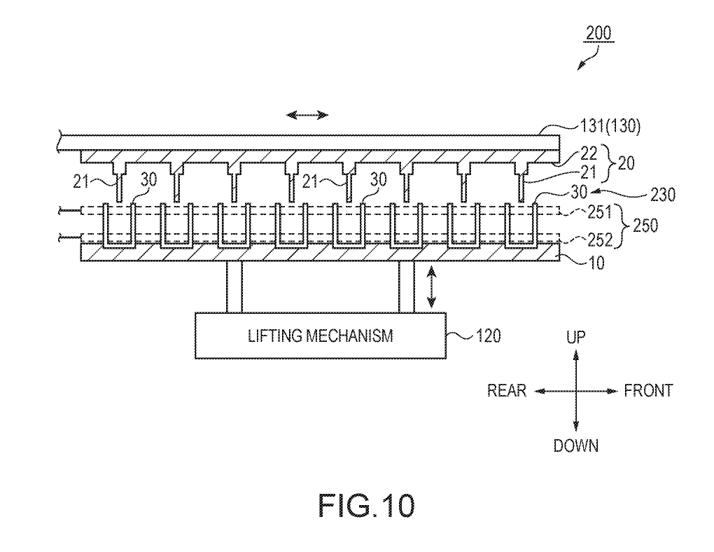

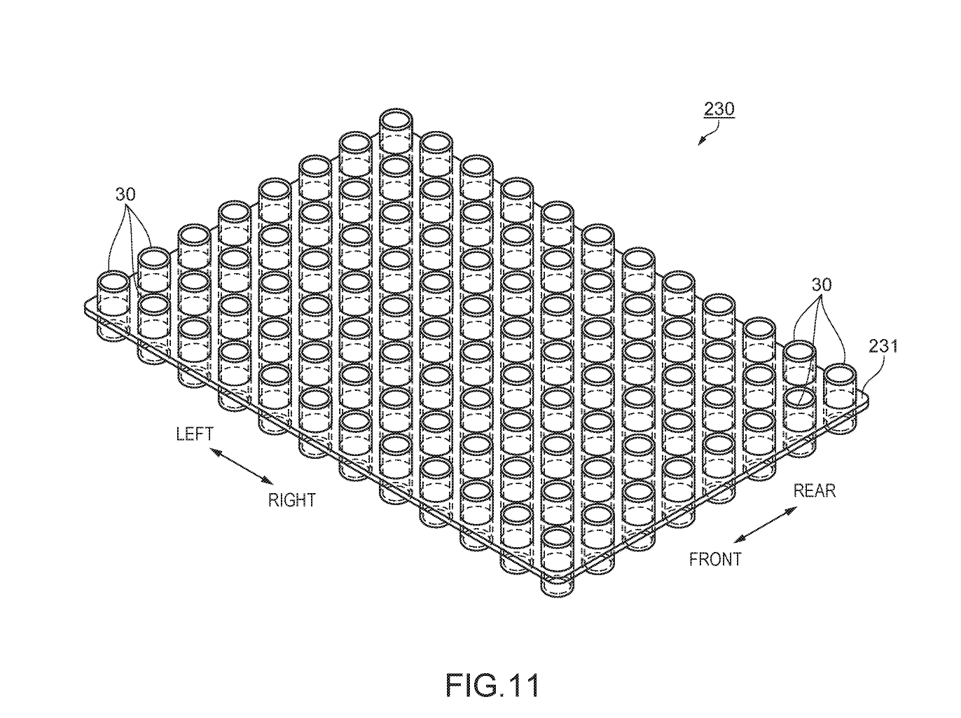

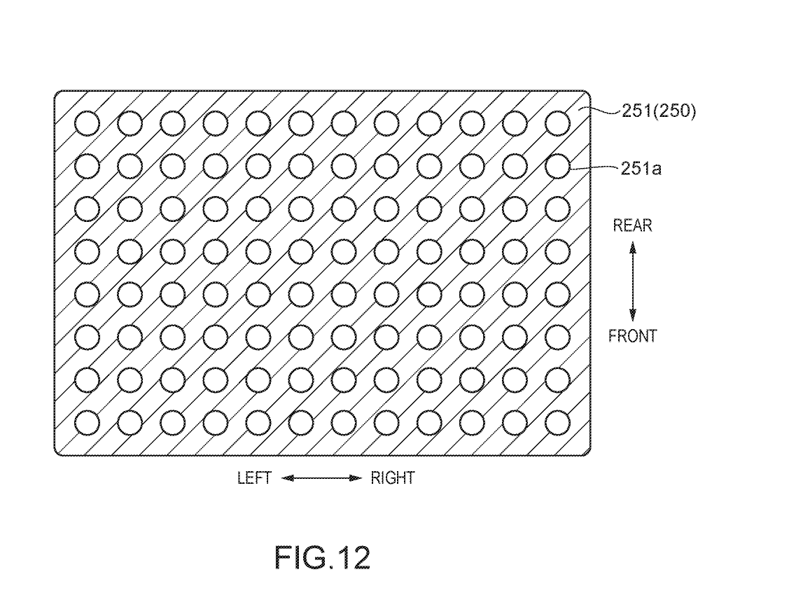

Next, a PCR device according to a second embodiment will be described with reference to FIGS. 10 to 12. FIG. 10 is a schematic view showing the PCR device according to the second embodiment, FIG. 11 is a schematic perspective view showing a vessel to be used in the PCR device according to the second embodiment, and FIG. 12 is a schematic plan view showing a heating section to be used in the PCR device according to the second embodiment. Hereinafter, in the description of the PCR device according to the second embodiment, the same reference numerals are assigned to basically the same components as those of the PCR device 100 according to the first embodiment, and the detailed description thereof will be omitted.

As shown in FIG. 10, a PCR device 200 of this embodiment includes a lower electrode 10, an upper electrode 20, a treatment chamber (not shown), a lifting mechanism 120, a moving mechanism 130, an electric field generation section (not shown), a heating section 250, an operation section (not shown), and a control section (not shown) in the same manner as the PCR device 100 of the first embodiment.

The lower electrode 10 can mount a vessel plate 230 including a plurality of vessels 30. In other words, the lower electrode 10 is electrically and mechanically provided in common for the plurality of vessels 30. Also the upper electrode 20 is electrically and mechanically provided in common for the plurality of vessels 30, and a columnar electrode section 21 is provided for each of the plurality of vessels 30.

The lifting mechanism 120 can move the lower electrode 10 having the vessel plate 230 mounted thereon up and down in the up-and-down direction, and can adjust the interelectrode distance between the lower electrode 10 and the plurality of columnar electrode sections 21. The moving mechanism 130 moves a support section 131 to which the upper electrode 20 is attached in the front-and-rear direction.

The heating section 250 includes a first heating section 251 and a second heating section 252 capable of heating the plurality of vessels 30 in common. The first heating section 251 is disposed on a side near the upper electrode 20 (columnar electrode section 21) with respect to the vessel plate 230, and the second heating section 252 is disposed on a side near the lower electrode 10 with respect to the vessel plate 230.

As shown in FIG. 11, the vessel plate 230 has a structure in which, for example, twelve vessels 30 in the right-and-left direction, eight vessels 30 in the front-and-rear direction, and a total of ninety-six vessels 30 are disposed at equal intervals and are integrated by a rib 231. Examples of the vessel plate 230 having such a structure include a plate obtained by molding using a plastic such as polypropylene. The number of the vessels 30 in the vessel plate 230 is not limited to 96.

As shown in FIG. 12, the first heating section 251 of the heating section 250 is, for example, a block heater having a rectangular outer shape, and has a plurality of holes 251a into which the plurality of vessels 30 can be inserted. The holes 251a are provided at equal intervals in the right-and-left direction and in the front-and-rear direction, respectively. Although not shown in the drawing, also the second heating section 252 is configured in the same manner as the first heating section 251. For example, when the lower electrode 10 is formed into a flat plate shape and is integrated with the second heating section 252, by the second heating section 252, the plurality of vessels 30 can be easily positioned and mounted on the lower electrode 10. In addition, it is also possible to dispose the first heating section 251 such that the first heating section 251 is supported by the rib 231 by adjusting and setting the height of the rib 231 in the vessel plate 230 in advance. That is, the vessel plate 230 may be configured such that it can determine the position of the first heating section 251 in the up-and-down direction.

According to the PCR device 200 of this embodiment and the PCR method using this device, a lot of reaction mixtures 60 can be simultaneously subjected to PCR in a shorter time as compared with the related art. The reaction mixtures 60 to be placed in the plurality of vessels 30 may contain a target nucleic acid of the same type or may contain a target nucleic acid of a different type. That is, it is possible to provide the PCR device 200 which can realize high productivity in the PCR process and a PCR method using this device.

### Third Embodiment

### Another PCR Method

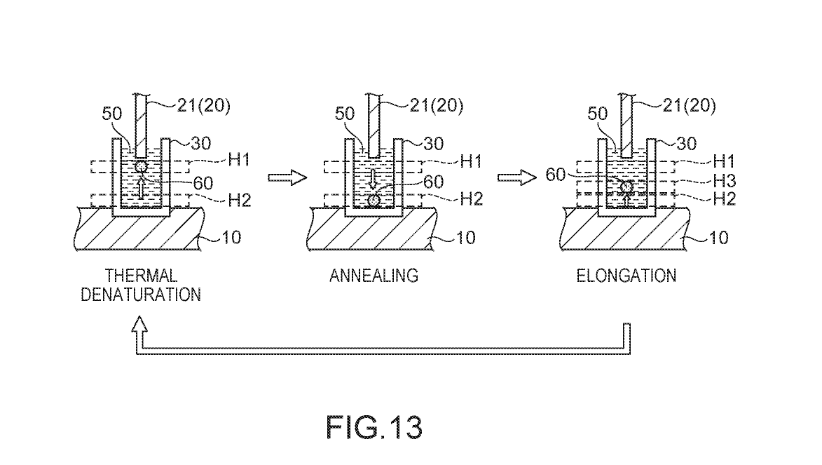

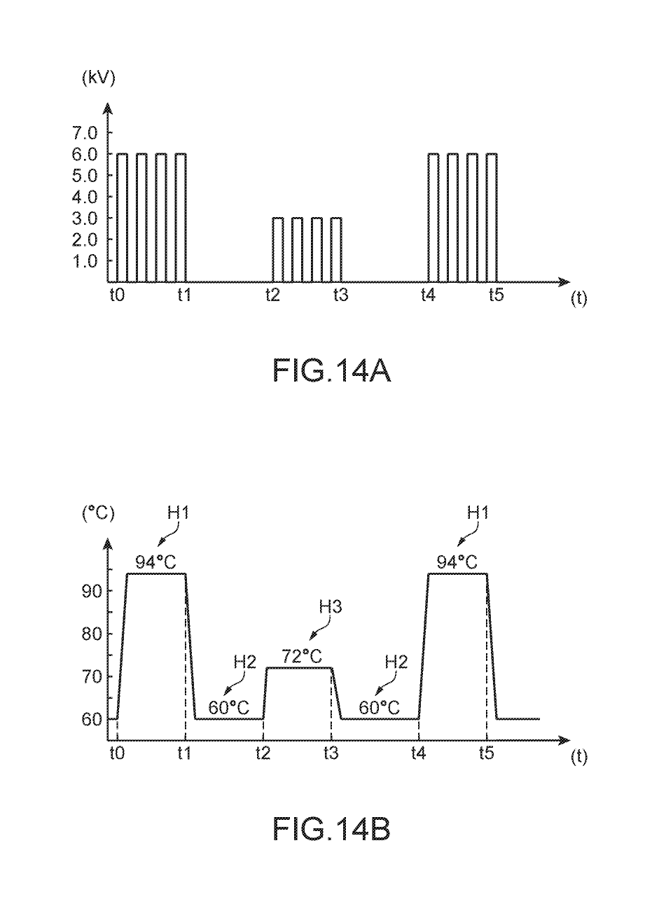

Next, another PCR method as a third embodiment will be described with reference to FIGS. 13, 14A, and 14B. FIG. 13 is a schematic view showing steps of another PCR method, FIG. 14A is a view showing a waveform of an alternating potential to be applied to an upper electrode in another PCR method, and FIG. 14B is a graph showing one example of a change in the temperature of a reaction mixture in another PCR method.

Another PCR method of this embodiment is a method which can be performed using the above-mentioned PCR device 100 (or the PCR device 200), and is characterized in that the temperature in the annealing reaction and the temperature in the elongation reaction are set to be different from each other.

Another PCR method of this embodiment includes a filling step (first step) of filling a vessel 30 with a liquid 50 and a reaction mixture 60, a heating step (second step), a thermal denaturation step (fourth step), an annealing step (fifth step), an elongation step (sixth step), and a step of repeating these steps in this order (third step) in the same manner as in the above-mentioned first embodiment. The steps from the filling step (first step) to the annealing step (fifth step) are the same as in the above-mentioned first embodiment, and therefore, hereinafter, the elongation step (sixth step) will be described.

In the elongation step (sixth step), as shown in FIG. 13, a control section 170 drives and controls an electric field generation section 140 to generate an electric field between a lower electrode 10 and a columnar electrode section 21 of an upper electrode 20 so that the reaction mixture 60 having undergone the annealing step is positioned in a third region H3 between a first region H1 and a second region H2 in the vessel 30.

The temperature of the first region H1 (upper part) of the liquid 50 in the vessel 30 is brought to a first temperature. The temperature of the second region H2 (lower part) of the liquid 50 in the vessel 30 is brought to a second temperature which is lower than the first temperature. The temperature of the third region H3 of the liquid 50 in the vessel 30 is brought to a third temperature which is an intermediate temperature lower than the first temperature and higher than the second temperature. In other words, the third temperature of the third region H3 can be arbitrarily set between the second temperature and the first temperature, and a third heating section for bringing the third region to the third temperature is not necessarily provided. In order to more accurately realize the third temperature, the third heating section may be provided.

In the annealing step (fifth step), as described above, for example, a primer 62 is bound to a single-stranded DNA 61a obtained in the thermal denaturation step at the second temperature which is lower than the first temperature. In the elongation step (sixth step) in another PCR method of this embodiment, a complementary strand to the single-stranded DNA 61a is synthesized at the third temperature using a heat-resistant enzyme 64 as the catalyst and also using a DNA synthesis substrate 63 with the primer 62 as the origin, whereby a double-stranded DNA 61 is formed. Depending on the type of the heat-resistant enzyme 64, the activity thereof at the third temperature is sometimes higher than the activity thereof at the second temperature. Therefore, it is preferred to perform the elongation reaction at the third temperature which is higher than the second temperature.

In the elongation step (sixth step), as shown in FIG. 14A, in a period between a time t₂ and a time t₃, for example, the lower electrode 10 is brought to a first potential (0 V) and an alternating potential (for example, 3 kV, at a frequency of 30 Hz) in which the potential changes between the first potential (0V) and a third potential which is higher than the first potential (0 V) and lower than the second potential (6 kV) is applied to the upper electrode 20. By doing this, an electric field generated between the lower electrode 10 and the columnar electrode section 21 is weaker than in the case where an alternating potential at 6 kV and at a frequency of 30 Hz is applied to the upper electrode 20. That is, a Coulomb force which acts on the reaction mixture 60 is decreased, and therefore, the reaction mixture 60 is positioned in the third region H3 between the second region H2 and the first region H1. In other words, the control section 170 drives and controls the electric field generation section 140 to generate an electric field between the lower electrode 10 and the upper electrode 20 (columnar electrode section 21) so that the reaction mixture 60 is positioned in the third region H3 (middle part) in which the temperature of the liquid 50 is brought to the third temperature. That is, according to the setting of the third temperature (position), the potential and the frequency to be applied to the upper electrode 20 may be adjusted.

By doing this, in the elongation step (sixth step), for example, as shown in FIG. 14B, in the period between the time t₂ and the time t₃, the temperature of the reaction mixture 60 can be maintained at the third temperature (for example, 72° C.) between the first temperature (for example, 94° C.) and the second temperature (for example, 60° C.).

In the elongation step (sixth step), a method in which the interelectrode distance is further decreased can also be exemplified as one example of the control method. However, since the third step in which the cycle from the thermal denaturation step (fourth step) to the elongation step (sixth step) is repeated about 50 times is performed, the lifting mechanism 120 is frequently driven and controlled to move the lower electrode 10 up and down, and therefore, the driving and controlling operation in the PCR device 100 becomes complicated. In terms of this, the driving and controlling operation can be more easily performed when the alternating potential to be applied to the upper electrode 20 is changed.

According to another PCR method using the PCR device 100 of this embodiment, in the elongation step (sixth step), the elongation reaction can be allowed to proceed at the third temperature at which the heat-resistant enzyme 64 favorably acts as the enzyme, and therefore, the time required for the elongation reaction can be reduced. That is, the target nucleic acid can be amplified while further reducing the time required for PCR.

The invention is not limited to the above-mentioned embodiments, and appropriate modifications are possible without departing from the gist or ideas of the invention readable from the appended claims and the entire specification. A PCR device thus modified and a PCR method using the PCR device are also included in the technical scope of the invention. Other than the above-mentioned embodiments, various modification examples can be made. Hereinafter, modification examples will be described.

### Modification Example 1

In the above-mentioned embodiments, an electric field is generated between the lower electrode 10 and the upper electrode 20 by applying an alternating potential to the upper electrode 20, however, the invention is not limited thereto. An electric field may be generated between the lower electrode 10 and the upper electrode 20 by applying a direct potential which is higher than the potential of the lower electrode 10 to the upper electrode 20.

### Modification Example 2

In the above-mentioned second embodiment, the lower electrode 10 and the upper electrode 20 are configured to be electrically and mechanically provided in common for the vessel plate 230 including a plurality of vessels 30, however, the invention is not limited thereto. For example, a configuration in which the lower electrode 10 is provided as a common electrode, and the columnar electrode section 21 is provided for each of the plurality of vessels 30 in an electrically and mechanically independent manner. According to this, even if the reaction mixture 60 of a different type is contained in the vessel 30, PCR can be performed under more efficient conditions by adjusting the potential to be applied to the columnar electrode section 21 or the interelectrode distance between the lower electrode 10 and the columnar electrode section 21 according to the type of the reaction mixture 60.

### Modification Example 3

In the PCR method of the above-mentioned embodiments, the target nucleic acid is not limited to a double-stranded DNA. For example, also in the case where a double-stranded RNA in which single-stranded RNAs are transcribed and bound to each other is amplified as the target nucleic acid, the PCR method of the above-mentioned embodiments can be applied.

### Modification Example 4

In the PCR method of the above-mentioned first embodiment, in the annealing step and the elongation step, the annealing reaction and the elongation reaction are performed in a state where the reaction mixture 60 is sunk in the liquid 50 and is positioned in the second region H2 without generating an electric field between the lower electrode 10 and the upper electrode 20 (columnar electrode section 21), however, the invention is not limited thereto. For example, the position of the second region H2 is set to a position above and slightly spaced apart from the lower electrode 10, and an electric field is generated by applying an alternating potential to the upper electrode 20 (columnar electrode section 21), and the annealing step and the elongation step may be performed in a state where the reaction mixture 60 is slightly floated by a Coulomb force. According to this, the annealing reaction and the elongation reaction can be performed in a state where the reaction mixture 60 is minutely vibrated and minutely stirred actively. That is, the time required for the annealing step and the elongation step can be reduced.

What is claimed is:

1. A polymerase chain reaction device, with which a nucleic acid contained in a reaction mixture placed in a vessel is amplified, comprising:
a lower electrode and an upper electrode disposed spaced apart from each other in the vertical direction;
an electric field generation section;
when the vessel is disposed between the lower electrode and the upper electrode,
a first heating section which heats the vessel on a side near the upper electrode;
a second heating section which heats the vessel on a side near the lower electrode; and
a control section, wherein
the vessel is filled with the reaction mixture and a liquid which has a lower specific gravity than the reaction mixture and is immiscible with the reaction mixture, and
the control section drives and controls the first heating section and the second heating section so that the liquid in an upper part in the vessel is brought to a first temperature and the liquid in a lower part in the vessel is brought to a second temperature which is lower than the first temperature, and also drives and controls the electric field generation section to generate an electric field between the lower electrode and the upper electrode so that the reaction mixture in a spherical shape in the liquid moves up and down repeatedly between the upper part and the lower part of the liquid by a Coulomb force due to the electric field.

2. The polymerase chain reaction device according to claim 1, wherein the control section drives and controls the electric field generation section so that a first potential is applied to the lower electrode, and when the reaction mixture is positioned in the upper part, an alternating potential, in which the potential changes between the first potential and a second potential which is higher than the first potential, is applied to the upper electrode.

3. A polymerase chain reaction device, with which a nucleic acid contained in a reaction mixture placed in a vessel is amplified, comprising:
a lower electrode and an upper electrode disposed spaced apart from each other in the vertical direction;
an electric field generation section;
when the vessel is disposed between the lower electrode and the upper electrode,
a first heating section which heats the vessel on a side near the upper electrode;
a second heating section which heats the vessel on a side near the lower electrode; and
a control section, wherein
the vessel is filled with the reaction mixture and a liquid which has a lower specific gravity than the reaction mixture and is immiscible with the reaction mixture, and
the control section drives and controls the first heating section and the second heating section so that the liquid in an upper part in the vessel is brought to a first temperature and the liquid in a lower part in the vessel is brought to a second temperature which is lower than the first temperature, and also drives and controls the electric field generation section to generate an electric field between the lower electrode and the upper electrode so that the reaction mixture in a spherical shape in the liquid repeatedly moves up and down to parts in the following order: the upper part, the lower part, and a middle part to be brought to a third temperature between the upper part and the lower part of the liquid by a Coulomb force due to the electric field.

4. The polymerase chain reaction device according to claim 3, wherein the control section drives and controls the electric field generation section so that a first potential is applied to the lower electrode, and when the reaction mixture is positioned in the upper part, an alternating potential, in which the potential changes between the first potential and a second potential which is higher than the first potential, is applied to the upper electrode, and when the reaction mixture is positioned in the middle part, an alternating potential, in which the potential changes between the first potential and a third potential which is lower than the second potential, is applied to the upper electrode.

5. The polymerase chain reaction device according to claim 1, wherein
a stage capable of mounting a plurality of vessels thereon is included, and
the lower electrode and the upper electrode are provided in common for the plurality of vessels.

6. The polymerase chain reaction device according to claim 5, wherein the upper electrode is provided for each of the plurality of vessels, and has a columnar electrode section capable of being inserted into the vessel.

7. The polymerase chain reaction device according to claim 5, wherein the stage and the lower electrode are integrated with each other.

8. The polymerase chain reaction device according to claim 1, wherein the lower electrode and the second heating section are integrated with each other.

9. The polymerase chain reaction device according to claim 1, wherein a lifting mechanism capable of adjusting an interelectrode distance between the lower electrode and the upper electrode by moving at least one of the lower electrode and the upper electrode is included.

10. A polymerase chain reaction method, with which a nucleic acid contained in a reaction mixture is amplified, comprising:
a first step of filling a vessel with the reaction mixture and a liquid which has a lower specific gravity than the reaction mixture and is immiscible with the reaction mixture;
a second step of heating an upper part of the liquid filled in the vessel to a first temperature at which the nucleic acid is thermally denatured, and also heating a lower part of the liquid filled in the vessel to a second temperature, which is lower than the first temperature, and at which the thermally denatured nucleic acid is amplified; and
a third step of moving the reaction mixture up and down repeatedly between the upper part and the lower part of the liquid by generating an electric field between the lower electrode and the upper electrode disposed spaced apart from each other in the vertical direction with respect to the vessel and allowing a Coulomb force to act on the reaction mixture in a spherical shape in the liquid.

11. The polymerase chain reaction method according to claim 10, wherein a first potential is applied to the lower electrode, and when the reaction mixture is positioned in the upper part, an alternating potential, in which the potential changes between the first potential and a second potential which is higher than the first potential, is applied to the upper electrode.

12. The polymerase chain reaction method according to claim 10, wherein
the reaction mixture contains a target nucleic acid, a nucleic acid synthesis substrate, a heat-resistant enzyme, and a primer, and
the third step includes a fourth step of thermally denaturing and separating the target nucleic acid into single-stranded nucleic acids at the first temperature, a fifth step of binding the primer to the single-stranded nucleic acid at the second temperature, and a sixth step of synthesizing a nucleic acid complementary to a single-stranded portion at the second temperature using the heat-resistant enzyme as the catalyst and also using the nucleic acid synthesis substrate with the primer bound to the single-stranded nucleic acid as the origin.

13. The polymerase chain reaction method according to claim 10, wherein
the liquid in the vessel has a middle part brought to a third temperature which is lower than the first temperature and higher than the second temperature between the upper part heated to the first temperature and the lower part heated to the second temperature,
the reaction mixture contains a target nucleic acid, a nucleic acid synthesis substrate, a heat-resistant enzyme, and a primer,
the third step includes a fourth step of thermally denaturing and separating the target nucleic acid into single-stranded nucleic acids at the first temperature, a fifth step of binding the primer to the single-stranded nucleic acid at the second temperature, and a sixth step of synthesizing a nucleic acid complementary to a single-stranded portion at the third temperature using the heat-resistant enzyme as the catalyst and also using the nucleic acid synthesis substrate with the primer bound to the single-stranded nucleic acid as the origin, and
the reaction mixture is repeatedly moved up and down to parts in the following order: the upper part, the lower part, and the middle part of the liquid by generating an electric field between the lower electrode and the upper electrode and allowing a Coulomb force to act on the reaction mixture in a spherical shape in the liquid.

14. The polymerase chain reaction method according to claim 13, wherein a first potential is applied to the lower electrode, and when the reaction mixture is positioned in the upper part, an alternating potential, in which the potential changes between the first potential and a second potential which is higher than the first potential, is applied to the upper electrode, and when the reaction mixture is positioned in the middle part, an alternating potential, in which the potential changes between the first potential and a third potential which is lower than the second potential, is applied to the upper electrode.

15. The polymerase chain reaction method according to claim 12, wherein the target nucleic acid is a DNA.

16. The polymerase chain reaction method according to claim 12, wherein the target nucleic acid is a nucleic acid in which two single-stranded RNAs are bound to each other.
